(19) United States
(12) Patent Application Publication
(10) Pub. No.: US 2010/0160484 A1
(43) Pub. Date: Jun. 24, 2010

(54) XANTHENE-BASED PLASTICIZER OF RESINS AND POLYMERS

(76) Inventors: John G. MacDonald, Decatur, GA (US); Teuta Elshani, Roswell, GA (US); Hristo A. Hristov, Roswell, GA (US); Molly K. Smith, Atlanta, GA (US); Ilona F. Weart, Woodstock, GA (US)

Correspondence Address: KIMBERLY-CLARK WORLDWIDE, INC.;Tara Pohlkotte, 2300 Winchester Rd., NEENAH, WI, 54956, US

(21) Appl. No.: 12/340,452

(22) Filed: Dec. 19, 2008

(51) Int. Cl. A61K 47/32

(52) U.S. Cl. 523/118

(57) ABSTRACT

A composition of matter for increasing the relative flexibility and plasticity of crystalline or semi-crystalline resins and polymers is described. In particular, the compositions include xanthene or anthene-based molecular structures that can increase the plastic properties of curable resins or thermosetting polymers when incorporated into the polymer material. Certain uses for such a polymeric composition are also provided.

### FIELD OF INVENTION

The present invention relates to a composition of matter for increasing the flexibility and durability of crystalline or semi-crystalline resins and polymers. In particular, the invention pertains to the plasticizing effect of xanthene-based molecular structures on curable resins or thermosetting polymers when incorporated into the polymer material.

### BACKGROUND

The morphology of polymer molecules and ways that molecules are arranged in a solid are important factors in determining the properties of materials. From polymers that crumble to the touch because of their rigid or brittleness to those that exhibit good elastomeric properties, the molecular structure, conformation and orientation of polymers can have a major effect on the macroscopic properties of the material. The general concept of self-assembly enters into the organization of molecules on the micro and macroscopic scale as they aggregate into more ordered structures. Crystallization is an example of the self-assembly process, as is the organizational orientation of liquid crystals.

Conventional thermoplastic polymers, curable or thermosetting polymer resins and films, such as polypropylene, cyanoacrylates, or polystyrene, tend to be relatively and brittle. Manufacturers have over the years tried to develop or modify conventional thermoplastic materials to make them more pliable or “softer,” but few have had success. This need for a new material composition or method to modify the polymeric materials to increase their relative plasticity remains unsatisfied. The present invention provides a plasticizer composition to address this need.

### SUMMARY

The present invention pertains to a curable composition of matter having a semi-crystalline polymer and a compound with a xanthene-based molecular structure. The polymer has a minimal crystalline content of about 40% to about 55% by weight of the polymer. The compound with a xanthene-based molecular structure is present in an amount of up to about 4% or 5%. The composition exhibits a ratio in a range from about 1.3:1.8:1.0 to about 1.6:1.5:1.0, respectively of a mesophase: crystalline phase: amorphous phase when cured. It is believed that the xanthene molecular structure exhibits a major effect that inhibits the formation of crystalline solid or semi-crystalline mesomorphic state, which increases the amorphous nature of the solidified polymer. In other words, it contributes to a manifestation of classic plasticizer properties with a relatively higher percentage of an amorphous state. The presence of xanthene-based molecular structures can reduce the relative rigidity and brittleness of a piece of polymer substrate, and imparts a greater flexibility or pliability.

In another aspect, the invention pertains to a method of plasticizing a crystalline-phase-containing polymer. The method involves providing in a mixture a polymer with about 30% to about 70% crystallnlnity and a plasticizing agent having a xanthene-based molecular structure present in an amount of up to about 5 wt. %, but typically about 0.1 wt. % to about 2.2 wt. %, of total composition; agitating and heating the mixture to a temperature of up to about 95° C. or 100° C.; and then allowing the mixture to cool to about ambient room temperature. Depending on the nature of the polymer (e.g., melting or curing point), the mixture may be heated to a temperature between about 50° C. or 60° C. and about 70° C. or 85° C. The polymer initially can have a mesophase of about 50% or less.

The present invention also pertains to a flexible barrier coating for mammalian skin. The coating includes a crystalline or semi-crystalline polymer and a plasticizing agent having a xanthene molecular structure. The barrier coating exhibits a modulus of about 1.8×10⁸ Pa to about 5.5×10⁸ Pa. Such physical properties are beneficial when developing a coating for surfaces that tend to bend or flex, such as the skin of animals, in particular, mammalian skin to which product like a skin sealant is applied.

More typically, the coating has a modulus of about 2×10⁸ Pa to about 4×10⁸ Pa. Human test subjects reported a noticeable difference between the feel of a skin sealant containing a conventional cyanoacrylate composition and one containing the present modified formulation. The difference in tightness or clinging against the skin is measurable reduction in the degree to which the coating cracked and flaked during use.

### BRIEF DESCRIPTION OF THE FIGURES

FIG. 1 is an X-ray diffraction curve of a control sample of a semi-crystalline polymer material.

FIG. 2 shows an X-ray diffraction curve of a sample of the semi-crystalline polymer material of FIG. 1, as modified with a plasticizer agent containing xanthene or xanthene-based molecules.

FIG. 3 is a graph showing comparative stress-strain curves for a control, Sample 1-1, a sample that incorporates a plasticizer according to the present invention, Sample 1-3, and a comparative example, Sample 1-4.

FIG. 4 is a graph showing the stress-strain curve of Sample 1-3 (5000 ppm Orange 5).

FIG. 5 is a heating curve of Sample 1-1 (Clear material).

FIG. 6 is an X-ray diffraction curve of Sample 2-4 (IS Clear+Xanthone), as normalized.

FIG. 7 is a graph showing comparative stress-strain curves for a control (Sample 2-1) and two samples containing respectively xanthene molecules, Sample 2-2 (5000 ppm xanthenes) and xanthone molecules, Sample 2-4 (5000 ppm xanthone).

FIG. 8A is a Dynamic Mechanical Analysis (DMA) graph of a control sample of resin.

FIG. 8B is a DMA graph of a sample of the resin of FIG. 8A, but incorporating a xanthenes-based plasticizing agent according to the present invention.

FIG. 9 is a graph showing the stress-strain curves of two samples with methyl-cyanoacrylate base polymers.

FIG. 10 compares the stress-strain curves of two samples of a methyl cyanoacrylate base polymers, pure control and incorporating dichloro-fluorescein, and a sample of a butyl-cyanoacrylate base polymer incorporating dichloro-fluorescein.

### DETAILED DESCRIPTION OF THE INVENTION

### SECTION I.—DEFINITIONS

In general, the present invention pertains to thermoplastic polymer compositions that are modified with a plasticizing compound containing a xanthene or xanthene-based molecular structure.

As used herein, the term “curable polymer” or “thermosetting material” refers to an organic macromolecule composed of a large number of monomers, the monomers have molecular weight that may range from about 95 daltons to about 150,000 or 200,000 daltons, which softens when exposed to heat and returns to its original condition when cooled to room temperature, such as cyanoacrylates.

As used herein, a “plasticizer,” “plasticizing agent,” or “plasticizing compound” is an organic compound that is added to a curable resin monomer—which when cured forms a relatively high molecular weight polymer (i.e., ≧500 daltons, up to about 100,000 daltons), which can both facilitate processing and increase the flexibility of the final product by modifying the molecular bonds of the polymer. Typically, the polymer molecule is held together by secondary valence bonds. The plasticizer replaces some of these bonds with plasticizer-to-polymer bonds, thus aiding movement of the polymer chain segments.

As used herein, a “xanthene” or “xanthene-based” molecule refers to an unmodified xanthene molecule or a derivative compound with a xanthene ring structure, as shown below. Xanthene (CH₂(C₀H₄)₂O) (dibezopyran, tricyclic), a yellow organic heterocyclic compound, has the following chemical structure:

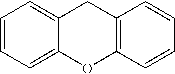

It is soluble in ether, and its melting point is 101-102° C. and its boiling point is 310-312° C. Xanthene is commonly used as a fungicide and is also a useful intermediate in organic synthesis. The xanthene molecule can be halogenated. Halogenated xanthene structures may include, for example, mono-bromo, di-bromo, tri-bromo, or tetra-bromo-fluorosceins; mono-fluoro, di-fluoro, tri-fluoro, or tetra-fluoro-fluorosceins; mono-iodo, di-iodo, tri-iodo, or tetra-iodo-fluorosceins; mono-chloro, di-chloro, tri-chloro, tetra-chloro-fluoroscein, and mixtures thereof. Additionally, mixed halogenated xanthenes structures such as tetra-bromo-tetra-chloro-xanthene (e.g., Drug and Cosmetic Red No. 27), are also contemplated.

### SECTION II.—DESCRIPTION

Although some polymers may be completely amorphous, the morphology of most polymers is semi-crystalline. That is, they form a combination of crystalline and amorphous portions with the amorphous regions surrounding the crystalline areas. The mixtures of small crystals and amorphous material melt over a range of temperature instead of at a single melting point. The crystalline material tends to have highly ordered and regular structures formed by folding and stacking of the polymer chains. The amorphous structure, in contrast, shows no long range order, and have molecular chains are arranged randomly and in long chains which twist and curve around one-another, making large regions of highly structured morphology unlikely.

The highly ordered crystalline structure and amorphous morphology of certain polymer materials determine the differing behaviors of the polymer. An amorphous solid is formed when the chains have little orientation throughout the bulk polymer. The glass transition temperature (T_{g}) is the point at which the polymer hardens into an amorphous solid. The glass transition temperature of a polymer is an important factor in its physical properties and behavior for certain desired uses. As the temperature of a polymer drops below its T_{g}, the polymer behaves in an increasingly brittle manner; while, as the temperature rises above the T_{g}, the polymer becomes more viscous-like. In general, polymers with T_{g} values of well below room temperature (˜20° C.) define the domain of elastomers, and those with values above room temperature define rigid, structural polymers.

The T_{g} can influence the mechanical properties of the polymeric material; in particular, the response of the material to an application of a force, namely: elastic and plastic behaviors. Elastic materials will return to their original shape once the force is removed. Plastic materials will deform fluidly and not regain their shape. In plastic materials, flow is occurring, much like a highly viscous liquid. Most materials demonstrate a combination of elastic and plastic behavior, exhibiting plastic behavior after the elastic limit has been exceeded. For example, polyvinyl chloride (PVC) has a T_{g} of 83° C. making it good, for example, for cold water pipes, but unsuitable for hot water. PVC also will always be a brittle solid at room temperature. Adding a small amount of plasticizer to PVC can lower the T_{g} to about −40° C. This addition renders the PVC a soft, flexible material at room temperature, ideal for applications such as garden hoses. A plasticized PVC hose can, however, become stiff and brittle in winter. In this case, as in any other, the relation of the T_{g} to the ambient temperature is what determines the choice of a given material in a particular application.

In the crystallization process, it has been observed that relatively short chains organize themselves into crystalline structures more readily than longer molecules. Therefore, the degree of polymerization (DP) is an important factor in determining the crystallinity of a polymer. Polymers with a high DP have difficulty organizing into layers because they tend to become tangled. Low molecular weight polymers (short chains) are generally weaker in strength. Although they are crystalline, only weak Van der Waals forces hold the lattice together. This allows the crystalline layers to slip past one another causing a break in the material. High DP (amorphous) polymers, however, have greater strength because the molecules become tangled between layers. In the case of fibers, stretching to 3 or more times their original length when in a semi-crystalline state produces increased chain alignment, crystallinity and strength.

Also influencing the polymer morphology is the size and shape of the monomers' substituent groups. If the monomers are large and irregular, it is difficult for the polymer chains to arrange themselves in an ordered manner, resulting in a more amorphous solid. Likewise, smaller monomers, and monomers that have a very regular structure (e.g. rod-like) will form more crystalline polymers.

The cooling rate also influences the amount of crystallinity. Slow cooling provides time for greater amounts of crystallization to occur. Fast rates, on the other hand, such as rapid quenches, yield highly amorphous materials. Subsequent annealing (heating and holding at an appropriate temperature below the crystalline melting point, followed by slow cooling) will produce a significant increase in crystallinity in most polymers, as well as relieving stresses.

In most polymers, the combination of crystalline and amorphous structures forms a material with advantageous properties of strength and stiffness. According to the present invention, while in furtherance of the work described in U.S. patent applications Ser. Nos. 11/974,369, and No. 11/974,393, the content of which are incorporated herein by reference, we have discovered that xanthene or xanthene-based compounds can impart significant plasticizing properties to a variety of crystalline or semi-crystalline in curable resins or polymer materials with a crystalline level of more than about 5% or 7%. Examples of suitable xanthene-based compounds include xanthene dyes (e.g., xanthene base structure of fluorescein systems). Xanthene dyes are a class of dyes which includes fluoresceins, eosins, and rhodamines. They fall into three major categories: the fluorenes or amino xanthenes, the rhodols or aminohydroxyxanthenes, and the fluorones or hydroxyxanthenes. Lillie, H. J. CONN'S BIOLOGICAL STAINS, p. 326 (Williams & Wilkins, 9th ed. 1977). Xanthene dyes tend to be fluorescent, yellow to pink lo bluish red, brilliant dyes. According to embodiments of the invention, xanthene and/or xanthene dyes can be incorporated into the thermoplastic polymer matrix by melt-mixing to enhance the physical plasticity of the resultant composition. The modulus of the fluorescein containing thermoplastic polymers are lower than those of the corresponding control thermoplastic polymers by about at least 5%-10%.

Nonetheless, according to the present invention, not all xanthene-based structures function well as a plasticizer. We have found that xanthenes-based compounds with ketone or carboxylic acid analogues (e.g., xanthone and xanthene-carboxylic acid) do not work as well as others since they appear not to impart good plasticizing characteristics, but rather can make the polymer material very brittle, even worse than a control sample of the original polymer material.

The present invention can be adapted for use with a variety of semi-crystalline resins and polymers. The present xanthenes-based plasticizer can function well to modify the modulus of curable polymers that have relatively small monomer units with a molecular mass of up to about 95,000 or 10,000 atomic mass units (daltons). The monomer units can have a molecular mass of as low as about 95 or 100 daltons. More particularly, the monomer units may range in mass from about 200 or 300 daltons to about 85,000 or 90,000 daltons (±200-500 daltons). Typically, the monomer unit are about 500 daltons to about 70,000 daltons inclusive (e.g., ˜750-60,000 daltons, 900-50,000 daltons, or desirably about 1,000 to about 30,000 daltons). More typically, the monomer molecule may be in a mass range from about 2,000 or 5,000 daltons to about 17,000 or 20,000 daltons.

The present invention relates to a curable composition of matter comprising a semi-crystalline polymer with a minimal crystalline content of about 40% to about 55% by weight of the polymer, and a compound with a xanthene-based molecular structure in an amount of less than 2%. The composition exhibits a ratio of about 1.3:1.8:1.0 to about 1.6:1.5:1.0, respective of a mesophase:crystalline phase:amorphous phase when cured. In certain embodiments, the curable composition exhibits a ratio of about 1.45:1.64:1.0, respective of said mesophase:crystalline phase:amorphous phase when cured. The polymer contains a crystalline content of about 35% to about 45% crystalline phase, 35% to about 45% mesophase, about 23% to about 27% amorphous state. The mesophase and said crystalline phase are each reduced by an amount of about 10-50% relative to the percentage of mesophase and crystalline phase of an identical composition absent the compound with xanthene-based molecular structure.

The compound with a xanthene-based molecular structure is present in the polymer matrix in an amount of about 0.01 wt. % up to about 2.0 wt. %. Typically, xanthene molecules or compounds with a xanthenies-based molecular structure are present from about 0.03 or 0.04 wt. % up to about 1.7 or 1.8 wt. %. More typically, the compound with a xanthene-based molecular structure is present at about 500 ppm (0.05 wt. %) to about 5000 ppm (0.5 wt. %). The semi-crystalline polymer has a vinylic functionalized monomer selected from: acrylate, cyanoacrylate, methacrylate, or styrene. In particular embodiments, the semi-crystalline polymer is a copolymer derived from one or more cyanoacrylate monomers or a blend of cyanoacrylate monomers. The cyanoacrylate can be an alkyl cyanoacrylate, wherein the alkyl group includes an ethyl, butyl, or propyl group. More specifically, the cyanoacrylate monomers may be selected from alkyl 2-cyanoacrylate, alkenyl 2-cyanoacrylate, alkoxyalkyl 2-cyanoacrylate, or carbalkoxyalkyl 2-cyanoacrylate, wherein the alkyl group may have 1 to 16 carbon atoms and may be methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate and 2-propoxyethyl 2-cyanoacrylate.

The composition can be adapted to form a flexible barrier coating for a skin sealant. For example, the composition can be formed into a film with about 1 mm (±0.05 mm) thickness and cured, said composition exhibits, at a stress of 50,000 g/cm², a deformation of at least 40% greater than an identical composition absent said compound with xanthene-based molecular structure.

According to an alternate embodiment, the invention can be an article of manufacture comprising curable polymers or thermoplastics. The curable polymer has a semi-crystalline polymer matrix incorporating a plasticizer composed of at least a xanthene molecule or a compound with a xanthene-based molecular structure, which can be present at about 500 ppm (0.05 wt. %) to about 5000 ppm (0.5 wt. %). In certain examples, the semi-crystalline polymer is a vinylic functionalized monomer selected from: acrylate, cyanoacrylate, methacrylate, or styrene. The semi-crystalline polymer can be a copolymer derived from one or more cyanoacrylate monomers or a blend of cyanoacrylate monomers, wherein the cyanoacrylate monomers are selected from alkyl 2-cyanoacrylate, alkenyl 2-cyanoacrylate, alkoxyalkyl 2-cyanoacrylate, or carbalkoxyalkyl 2-cyanoacrylate, wherein the alkyl group may have 1 to 16 carbon atoms and may be methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate and 2-propoxyethyl 2-cyanoacrylate.

According to another aspect, the invention discloses a method of plasticizing a crystalline-phase-containing polymer. The method comprises: providing in a mixture a polymer with about 30% to about 70% crystallinity and a plasticizing agent having a xanthene-based molecular structure present in an amount of up to about 2.2 wt % or 2.4 wt %, more typically up to about 1.75 or 1.8 wt. %, of total composition; agitating and heating said mixture to a temperature of up to about 85° C.; and then allowing the mixture to cool to about ambient room temperature. Typically, the mixture is heated to a temperature of between about 50° C. and 80° C. (e.g., about 60° C. or 70° C.). Structurally, the polymer should contain a mesophase of greater than 33% or 35% of the polymer matrix. In certain curable polymner materials the mesophase can be between about 37% or 40% up to about 55% or 60% or 75%.

In yet another aspect, the present invention can be adapted to create a flexible barrier coating that can be applied to mammalian skin without the shortcomings of conventional films, such as cracking and spalling of an inelastic dried film layer when subjected to skin movement. The present barrier coating includes a crystalline or semi-crystalline polymer and a plasticizing agent having a xanthlene molecular structure, said barrier coating exhibiting a modulus of about 1.8×10⁸ Pa to about 5.5×10⁸ Pa. Typically, the flexible barrier coating has a modulus of about 2×10⁸ Pa to about 4×10⁸ Pa.

### SECTION III.—PRACTICAL APPLICATIONS

The present plasticizer material can be incorporated into the formulation of a variety of products that contain alkyl-cyanoacrylates, with an alkyl chain ranging from C2 to C12.

A. Methyl and butyl-cyanoacrylates

As illustrated in the accompanying figures, curable resins, such as methacrylates or epoxy materials, which are modified with pure xanthene or halogenated xanthene molecules exhibit relatively good resistance to stress-strain, behavior. Strain of a polymer sample is expressed as a percentage (x %) of a sample's original length dimension. The polymer sample modified with xanthene can withstand nearly twice the amount of strain as that experience by a control resin sample before it fractured. In other words, if the control sample is able to withstand up to about 5% or 6% strain before breaking, the xanthene-doped polymer sample is able to withstand up to about 10% to 12% strain before tearing. In contrast, curable polymer materials that are doped with ketone and carboxylic acid analogues of xanthene (i.e., xanthone, xanthenic acid) appear not to exhibit a similar enhanced plasticizing effect. A polymer sample incorporating xanthone molecules is only slightly better than the control sample in being able to adapt to a strain load before breaking. Moreover, the polymer sample incorporating xanthenic acid molecules become too brittle even to remove from the surface of a mold.

In certain examples, the polymer is an alkyl cyanoacrylate selected from a group including, for example alkyl 2-cyanoacrylate, alkenyl 2-cyanoacrylate, alkoxyalkyl 2-cyanoacrylate, and carbalkoxyalkyl 2-cyanoacrylate. The cyanoacrylates also may be selected from, for instance, methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate and 2-propoxyethyl 2-cyanoacrylate. The alkyl group of the cyanoacrylate has 1 to 16 carbon atoms. More desirably the alkyl group has 1 to 6 or 8 carbons. It is observed that unmodified xanthene molecules can reduce the relative melting point for both the crystalline phase and mesophase of the polymer, which results in an extension of the plasticizing effect.

B. Skin Study and Elastic Modulus of Skin

An application of the present invention can be in the healthcare or medical arena. In recent years, coatings containing cyanoacrylates have been developed to help reduce the incidence of surgical site infections. The coating is applied to a clean area of a patient's skin to immobilize microbes that may be present before the surgeon makes an incision through the coated area. An example of a composition for such a coating or a skin sealant is detailed in Table 1, below. Unfortunately, because of the rigid nature of cyanoacrylate films, the skin sealant tends typically to become inflexible and brittle when dried on the patient's skin. When encountering the natural bending and flexing of the body, the brittleness of the coating causes the coating to easily crack and spall off.

TABLE 1
Skin Sealant Formulation

| Formulation Component | Function in Formulation | Amount Present |
|---|---|---|
| n-butyl cyanoacrylate | monomer | 80% |
| tributyl-o-acetyl citrate | plasticizer | 20% |
| butylated hydroxyl toluene | stabilizer and free | 500 ppm |
| | radical inhibitor | |
| sulfur dioxide | stabilizer and anionic | 150 ppm |
| | inhibitor | |
| D&C violet #2 | colorant | 100 ppm |
| water | | 500 ppm or less |

An unexpected benefit of incorporating xanthenes dyes, which provide a colorant that would allow visual indication to a user for both coverage and cure of the skin sealant, is that, when cured the cyanoacrylate film exhibited a greater degree of flexibility than an identical cyanoacrylate composition absent the xanthene dye. The greater flexibility of the polymer film leads to reduced cracking and flaking of the cured skin sealant.

In particular embodiments, the plasticizer may be incorporated in a polymeric skin sealant for surgical or other applications. An example of such a sealant is INTEGUSEAL™, which is commercially available from Kimberly-Clark Corporation. INTEGUSEAL™ contains alkyl-cyanoacrylate compounds or adhesives such as described in U.S. Pat. Nos. 6,136,326, 6,224,622, or 6,281,310, all to D. L. Kotzev, the contents of which are incorporated herein by reference.

INTEGUSEAL™ (C4 alkyl-cyanoacrylate composition) contains a cyanoacrylate resin with 20% tributyl o-acetylcitrate (TBAC) plasticizer. Even with this level of plasticizer cracking and flaking still occurs due the crystalline nature of the acrylate resin. When xanthene dyes are introduced, according to the invention, to this formulation in parts-per-million (ppm) levels there is a significant improvement in the flexibility of the resultant film. This has been measured and quantified by x-ray analysis and thermal mechanical analysis (TMA). (See Empirical section below.) In testing on skin this improvement in properties was observed in decrease cracking and flaking along with a more comfortable feel while on the skin (less pulling or tighting of the skin).

In clinical trials, human volunteers had a coating of INTEGUSEAL™ applied onto their bare backs. The volunteers clearly perceived a difference between a current, commercially available INTEGUSEAL™ containing Drug & Cosmetic (D&C) violet 2 and an experimental analogue formulation containing dibromofluorescein (DBF). The volunteers commented that the commercial formulation felt like superglue on skin, which did not flex as the test subjects moved or bent their bodies. The commercial formulation pulled against the skin slightly, giving a tighter feel to the coating. In contrast, the volunteers felt that the DBF-containing formulation to be less “clingy” or “tight” against their skin and it appeared to be more flexible with greater elasticity. Although these observations are qualitative, all volunteers observed and remarked on this sensation.

The visco-elastic properties of human skin have been reported in the literature. It is a difficult system to measure due to the property is due to a combination of the components of human skin (collagen, elastin and ground substances). Results indicate that maximum thickness of human skin is reached when an individual is around the age of 40 years. Standardized skin extensibility decreases with age and has to be considered in relation to the total water content. The literature has several studies to determine the elastic modulus of human skin; the values obtained cover a range which was attributed to the particular method of analysis used. Of specific interest was the method of Graham and Holtz which obtained a value of 1.8×10⁷ Pa (Pascal). The current INTEGUSEAL™ coating has an elastic modulus of 7×10⁸ Pa when applied and cured on a person's skin. This formulation can be reduced by the addition of the DBF to about 4×10⁸ Pa. This result represents a relative decrease of about 43%. Other examples can range between about 20-45%. The addition of the DBF shifts the elastic modulus towards that of human skin, which was sensed by the volunteers. This advantageous feature allows for development of skin sealant compositions that can exhibit an elastic modulus approaching the elastic modulus of human skin.

Using 4,5-dibromofluorescein (Aldrich Chemical Co., Milwaukee Wis.) dissolved into an INTEGUSEAL™ formulation at 5000 ppm concentration. A 500 ppm sample was also made. Films are made by drawing down a thin film of the various formulations including a control. The samples were then twisted repeatedly 50 times. The control was badly cracked and flaked, while the DBF samples, in contrast, remained pliable and unchanged. When samples were placed onto the skin of a human volunteer at a moving part of the arm and allowed to cure. The volunteer felt a clear distinction on the skin between the two samples. Reporting that is was not as “clingy” compared to the control when the arm moved. Later on x-ray and TMA analysis measured and documented the difference in morphology of the sample and control.

X-ray diffraction (XRD) results show that the dry INTEGUSEAL™ materials contain a phase with intermediate atomic order—mesophase. To this end Drug & Cosmetic (D&C) Orange 5 was identified to provide a liquid coating that was bright fluorescent yellow when applied turning to a fluorescent coral pink when the coating was cured. It appears that the pigment Orange 5 reduces the mesophase levels, which affects the mechanical properties of the dry films.

The incorporation of a xanthene-based structure in the normally rigid polymer matrix increased by at least 10% the amount of amorphous content in the polymer. The plasticizing agent also decreases the relative amount of mesomorphic state in the polymer, in which a significant fraction of has a molecular arrangement intermediate between crystalline solid and amorphous phases, which under X-ray diffraction analysis appears like “liquid crystals.”

### SECTION IV.—EMPIRICAL EXAMPLES

This section describes the experiments to investigate xanthene compounds to determine the role of the substituents and the structure itself plays on this plasticizer effect.

Alkyl cyanoacrylates [R(COR′)C═CH2] where the alkyl group is between C1 to C12. Monomer molecular weights (MW) range from about 86 to about 192.

Alkyl cyanoacrylates are used as “Superglue” (ethyl and methyl cyanoacrylates), skin sealants (butyl cyanoacrylates), and surgical suture and organ repair adhesives (octyl cyanoacrylates).

Alkyl cyanoacrylates cure to become solids quite efficiently resulting in a solid substance with very high molecular weight where all the monomer is polymerized/crosslinked. The final molecular weight of the cured substance depends on the amount of monomer used or present at the beginning, therefore it is difficult to give a final molecular weight as it depends on the amount of monomer used.

### Experiment 1.

### Material Samples

Sample 1-1—Clear cyanoacrylate
Sample 1-2— INTEGUSEAL™ Material+500 ppm Orange5
Sample 1-3—INTEGUSEAL™ Material+5000 ppm Orange5
Sample 1-4—INTEGUSEAL™ Material+Violet
   The liquid materials were spread on microscopic slides. After drying at room temperature, the resulting films were removed with razor blade and analyzed.

### Differential Scanning Calorimeter (DSC)

The samples were analyzed on a TA Instruments DSC 2920 Modulated DSC (Standard Cell) using the following experimental procedure: Approximately 5 mg, cut from a random place of the respective materials, were run in the temperature interval −125° C. to 220° C. with a heating/cooling rate of 10° C./min in an inert gas (N₂) atmosphere.

### X-Ray Diffraction (XRD)

The film samples were analyzed on an X-ray diffractometer D-max Rapid from Rigaku Corp. equipped with a two dimensional (2-D) position sensitive detector. The measurements were executed in transmission geometry and Cu Kα radiation (λ=1.5405 Angstrom). The results were corrected for background and air scattering.

### Dynamic Mechanical Analysis (DMA)

The film samples with thicknesses in the range 30μ-60μ were analyzed on a Rheometrix Solids Analyzer DMTA V. The measurements were executed at room temperature in a frequency sweep mode (1 Hz to 10 Hz) by increasing the loads until the failure of the materials.

Samples are analyzed using X-ray diffraction, and exhibited three large intensity peaks representing the three phases of the polymer material. A crystalline phase is represented by an intensity peak between about 17-20, an amorphous phase is represented by a peak in the range of about 10-17, and a mesophase is a peak in a range of about 4-8.

Comparison of the accompanying X-ray diffraction curves for an experimental polymer sample and a control sample containing the xanthenes-based plasticizing agent shows that the amorphous concentration increases from an original intensity of about 4-6 counts in the control to about double at 10-12 counts in the experimental sample. Correspondingly, incorporation of a xanthene-based compound in the polymer results in a decrease of the mesophase content by about 5-7 or 10 units. The crystalline phase and mesophase of the polymer each is reduced by about 20%, 22%, or 25% up to about 45% or 50%. The X-ray diffraction intensity of the crystalline phase is reduced by about 3,000 to about 5,000 counts and mesophase by about 6,000 to about 10,000 counts.

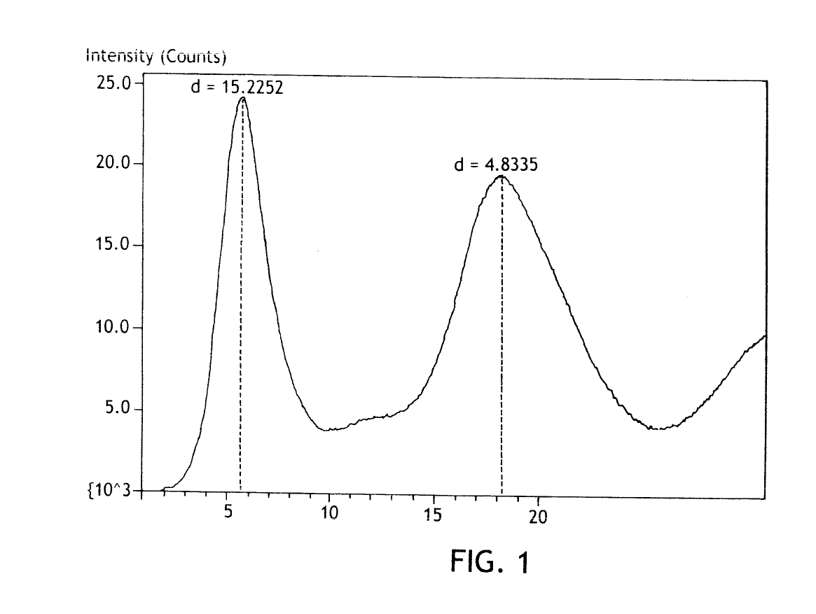

FIG. 1 shows the X-ray diffractogram of Sample 1-1. The curve is characterized by a diffraction peak located at diffraction angle ˜5.7°2θ, corresponding to d-spacing of ˜15 Angstroms (1.5 nm). A broad amorphous halo is located at diffraction angle ˜18.5°2θ. Assuming that the peak at diffraction angle ˜5.7°2θ, comes from a crystalline substance, one can compute a crystal size in the range of 3 nm. It should be noted that crystal sizes measured in common polymeric materials are larger than 5 nm. This fact and taking into account the lack of higher order crystalline reflections indicate that the INTEGUSEAL™ material is non-crystalline. On the other hand it is evident that the atomic order is more pronounced than in typical amorphous material. A coherent explanation of the XRD results is the assumption that the dry INTEGUSEAL™ material contains a mesomorphic phase (meso-phase), which is intermediate between the crystalline and amorphous phases.

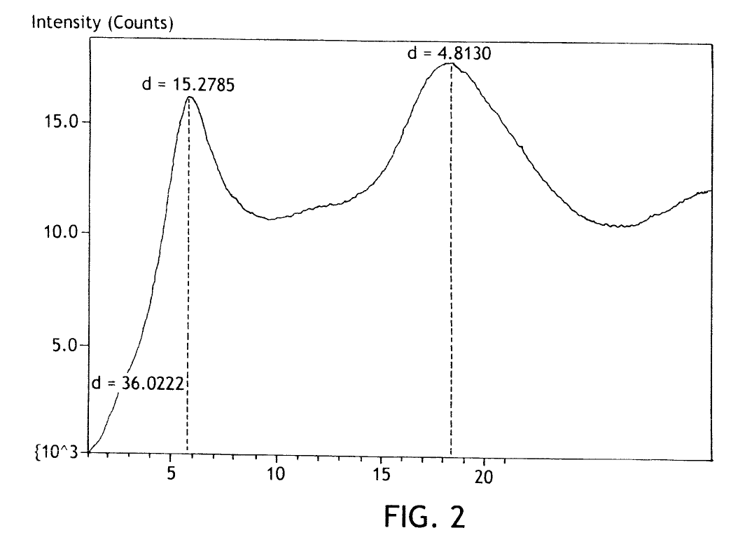

An inspection of FIG. 2 (Sample 1-3—INTEGUSEAL™+5000 ppm orange5) indicates that this material also contains mesophase. It appears, however, that the intensity of the diffraction peak at angle ˜5.7°2θ is lower than the counterpart in FIG. 1. Our measurements showed that the XRD curve of Sample #4 (INTEGUSEAL™+violet) is similar to the curve in FIG. 1, while the curve of Sample 1-2 (INTEGUSEAL™+500 ppm) is similar to the curve in FIG. 2.

TABLE 2 summarizes the ratios of the intensities of the mesomorphic peaks divided by the intensities of the respective amorphous halos.

TABLE 2

| | | | Elongation |
|---|---|---|---|
| | Ratio | | at |
| Sample ID | Int_{5.7}/Int_{18.5} | E′ [dyn/cm²] × 10⁹ | Break [%] |
| Sample 1-1 - Clear | 1.3 | 7 | 7 |
| Sample 1-2 - 500 ppm | 0.75 | 4 | 10 |
| Orange 5 | | | |
| Sample 1-3 - 5000 ppm | 0.6 | 3 | 10.5 |
| Orange 5 | | | |
| Sample 1-4 - Violet | 1.2 | 6 | 8 |

From Table 2, one can see that the clear material (Sample 1-1) and the material containing violet pigment (Sample 1-4) are characterized with ratios larger than 1, while the materials containing the pigment Orange 5 exhibit ratios less than 1. This is an indication that the materials containing the Orange 5 additive exhibit a lower mesophase content in comparison to Samples 1-1 and 1-4.

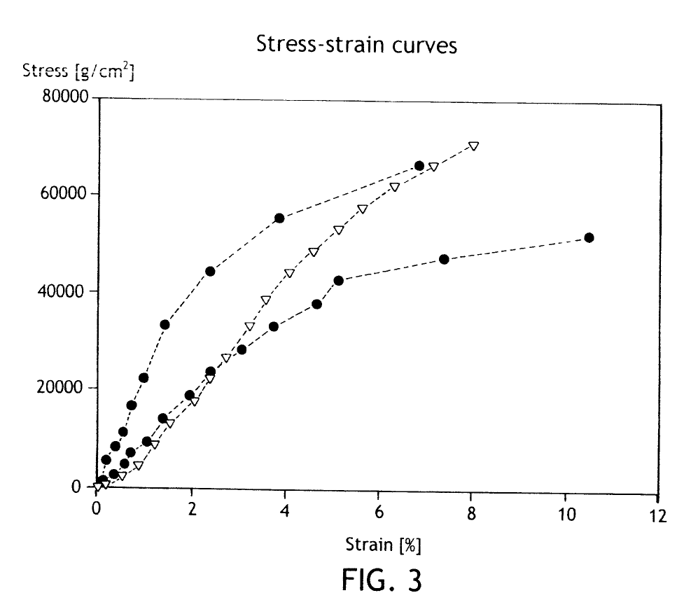

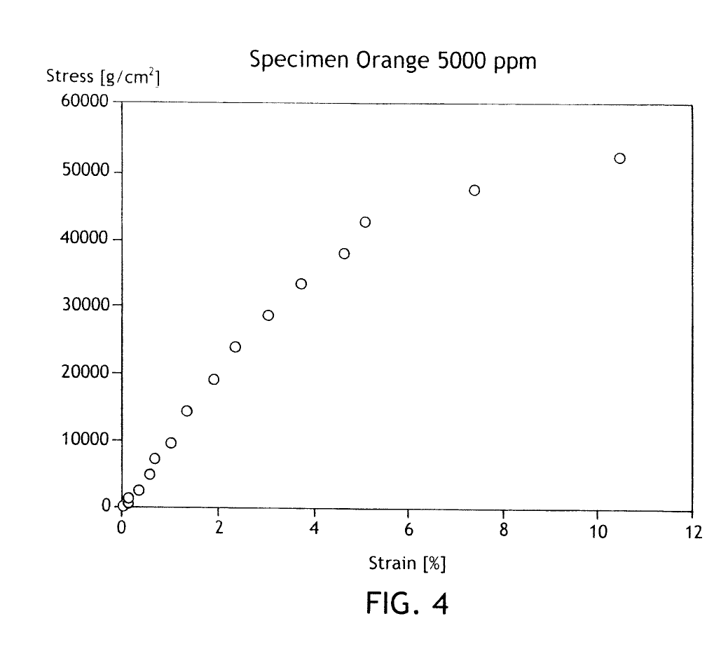

To investigate the possible effect of the mesophase content on the mechanical properties of the materials DMA tests were performed. In FIG. 3, the stress-strain curves for Samples 1-1 (black circles), 1-3 (open circles), and 1-4 (triangles) are shown together for ease of comparison. The stress-strain curve of Sample 1-3 alone is plotted in FIG. 4. By using the stress-strain curves it is straightforward to obtain the elongation at break and dynamic moduli (E′—measure of the rigidity of the materials). The DMA results are summarized in Table 2. The analysis of the results suggests that the reduced meso-phase levels produce softer and more pliable materials—lower E′ and higher elongation at break.

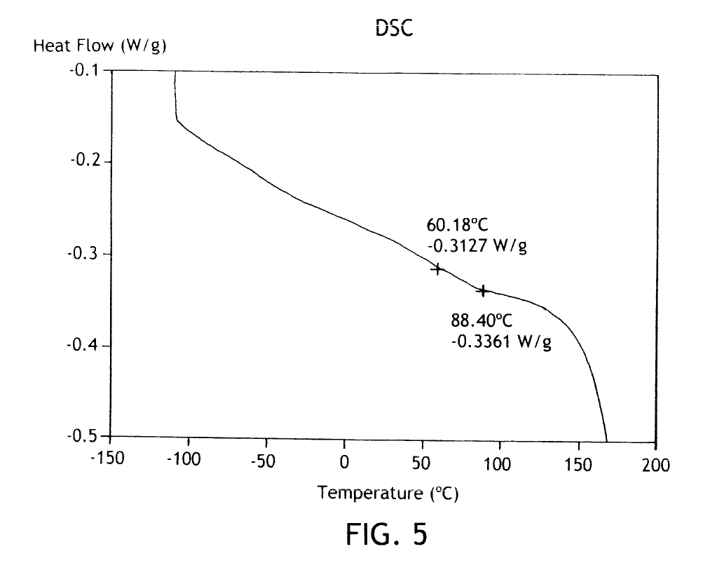

FIG. 5 shows a plot of the heating curve for Sample 1-1 (clear material). The results show small transitions at ˜60° C. and at ˜90° C. and rapid degradation of the material at temperatures higher than ˜150° C. The results from the rest of the sample materials are fairly similar to the results in FIG. 5; hence they are not reproduced here.

### Experiment 2.

### Material Samples

Sample 2-1—clear cyanoacrylate
Sample 2-2—cyanoacrylate+Xanthene 99% (5000 ppm)
Sample 2-3—cyanoacrylate+Xanthene-9-Carboxylic Acid (5000 ppm)
Sample 2-4—cyanoacrylate+Xanthone (5000 ppm)

Curable polymer resins in a liquid form are spread on microscopic slides. After drying at room temperature, the resulting films were removed with razor blade. Sample 2-3 was very brittle and developed small cracks in the process of removal from the microscopic slide; hence, DMA tests on Sample 3-3 were unsuccessful.

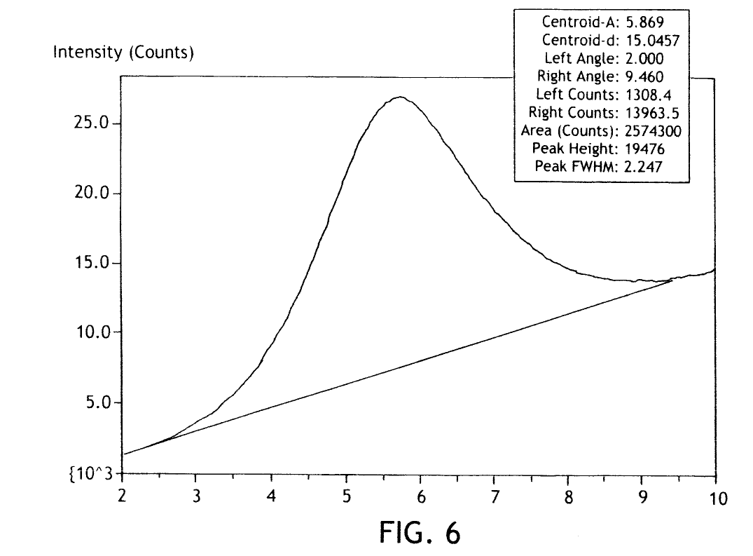

FIG. 6 is an X-ray diffraction curve of Sample 2-4 (IS Clear+Xanthone), after normalization. In FIG. 6, the intensity of the peak is at 4.2 Å (from the PE standard) was used to normalize the mesomorphic peaks at ˜15.4 Å. Under these conditions the area under the peak is proportional to the mesophase concentration. After normalization, the area under the mesomorphic peak for each of the samples (see FIG. 6) was computed and the results are summarized in TABLE 3. The data indicates that Sample 2-3, which exhibits the highest levels of mesophase, does not undergo increased plasticizing. In contrast, Sample 2-4 is characterized with the lowest mesophase concentration and does exhibit good plasticity. The difference between Samples 2-1 and 2-4 is less than 1%.

TABLE 3

| | | Int_{5.7} | | Elongation at |
|---|---|---|---|---|
| Sample ID | | ×10⁶ | E′ [dyn/cm²] × 10⁹ | Break [%] |
| Sample 2-1 | (Clear) | 4.1 | 6.7 | 5.1 |
| Sample 2-2 | (Sample 1 + | 5.7 | 6.5 | 9.5 |
| | Xanthene) | | | |
| Sample 2-3 | (Sample 1 + | 6 | N/A | N/A |
| | Xanthene + | | | |
| | Carboxylic | | | |
| | Acid) | | | |
| Sample 2-4 | (Sample 1 + | 3.8 | 4.5 | 5.3 |
| | Xanthone) | | | |

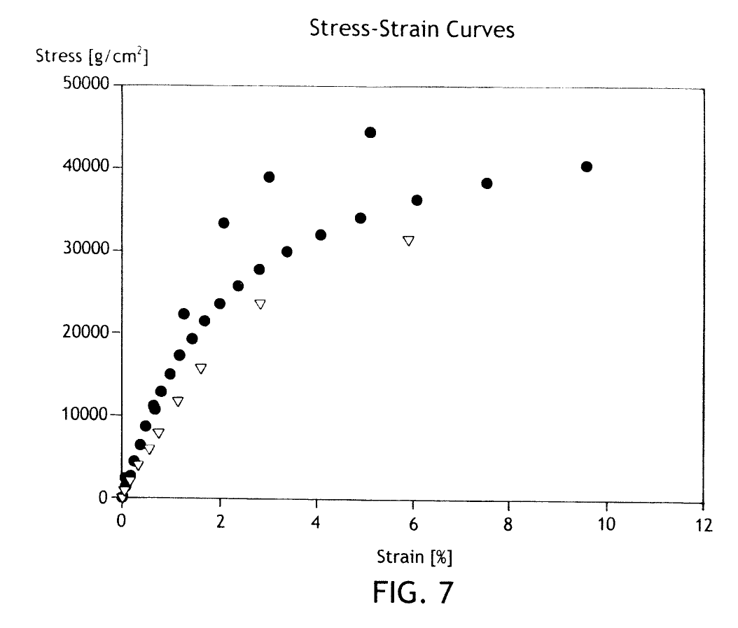

Using a DMA instrument, specimens are tested to ascertain the effect of additives on the mechanical response of INTEGUSEAL™ materials. In FIG. 7, stress-strain curves of the three materials are shown, and TABLE 3 summarizes the data for the respective dynamic moduli and elongations at break. The results show that the control clear material (Sample 1) exhibits the highest dynamic modulus and lowest elongation at break. The results for Sample 2-2 are unusual because it contains more mesophase than Samples 2-1 and 2-4, but it exhibits the highest elongation at break. Sample 2-3 was the most brittle material.

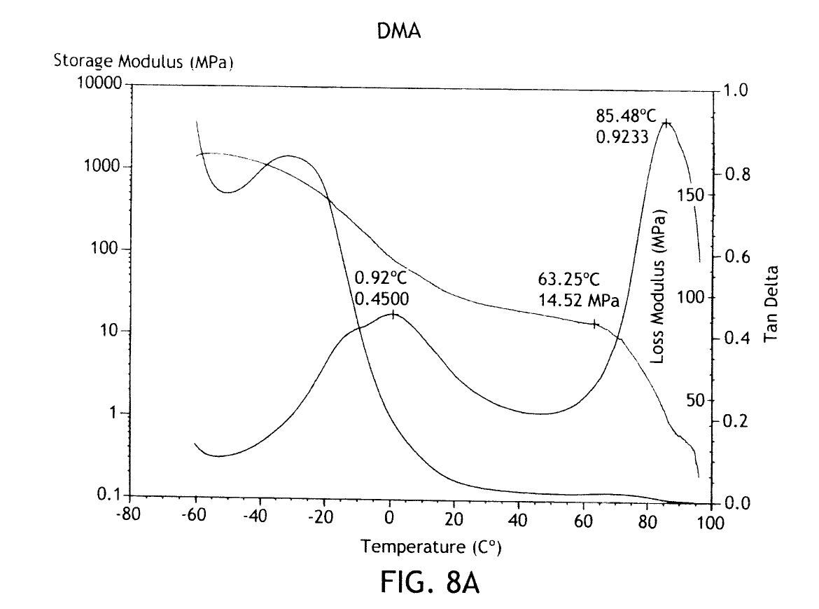

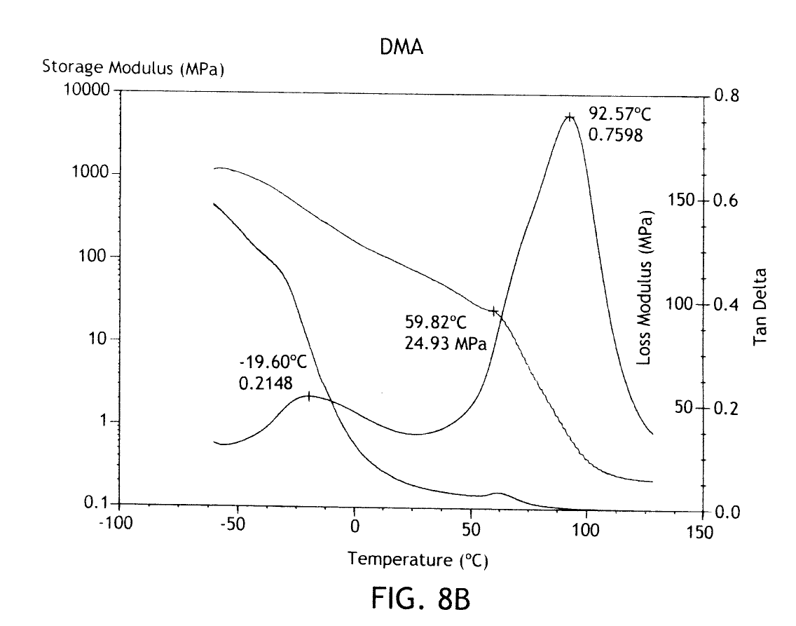

To obtain a better understanding of the differences in the phase structures of the INTEGUSEAL™ control, Sample 2-1, and INTEGUSEAL™ containing xanthenes, Sample 2-2, the two materials were tested in the temperature interval from −60° C. to +110° C., with a constant heating rate (2° C./min) and constant frequency (2 Hz) in tension/tension deformation mode. FIG. 8A—Temperature changes of the Storage Modulus (E′—green curve), Loss Modulus (E″—brown curve) and tan δ (E″/E′—blue curve); sample 2-1. FIG. 8B—Temperature changes of the Storage Modulus (E′—green curve), Loss Modulus (E″—brown curve) and tan δ (E″/E′—blue curve); Sample 2-2. As plotted in FIGS. 8A and 8B, the results show that the onset of the main glass transition event is ˜60° C. for both materials. The tail δ peaks are also fairly close (˜85° C. vs. ˜92° C.). There are also sub-ambient glass transitions and the tan δ peaks are located at different temperatures: ˜1° C. (Sample 2-1) vs. ˜−20° C. (Sample 2-2).

When dry, the INTEGUSEAL™ material is a multiphase material, as confirmed by XRD, and the main phase structural transition is ˜90° C. There are also secondary phases with lower temperature structural transitions. Apparently the presence of additives affects both the amount of secondary phases and their respective transition temperatures. In other words, although the xanthenes-containing sample has a greater amount of mesophase, the sample has a lower glass transition temperature resulting in a more flexible film compared to the control. Xanthene-based molecular structures, such as both xanthene and dibromofluorescein, are strong plasticizers of cyanoacrylates. In contrast, xanthone and xanthene-carboxylic acid do not show any plasticizing effect.

A comparison of the XRD and DMA results shows that a correlation between the mesophase content and the mechanical response. The polymer materials containing the highest levels of mesophase are more brittle than the specimens containing xanthene-based molecular structures. The results show that a polymer having a crystallinity content of 45%, such as a cyanoacrylate polymer solid by itself or containing violet 2 exhibited the most brittle physical properties—highest dynamic modulus and shortest amount of elongation at break. Conversely, a cyanoacrylate solid containing 4′,5′-dibromofluorescein (DBF) exhibits the most ductile behavior—lowest dynamic modulus and greatest amount of elongation at break; hence, 4,5′-dibromofluorescein is a strong plasticizer of cyanoacrylate formulations.

### Experiment 3.

Xanthene show good plasticizing properties in various kinds of cyanoacrylate resins.

### EXAMPLE

### Incorporating Xanthenes in Methyl Cyanoacrylates

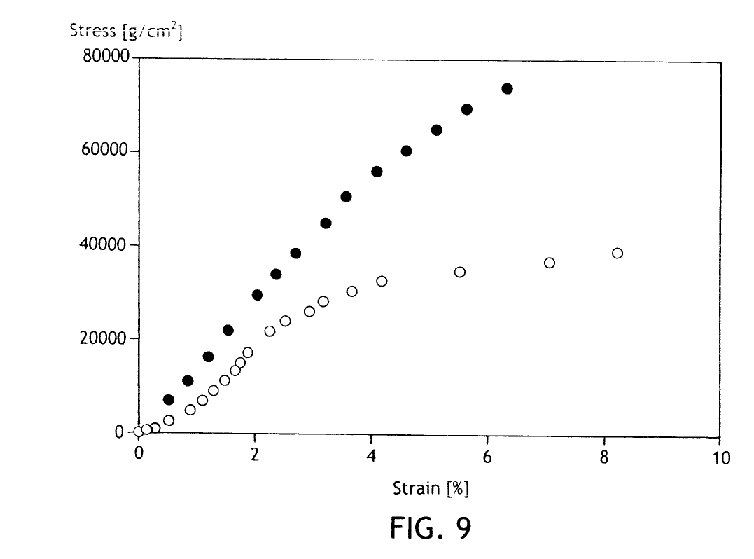

We blended dichlorofluorescein into a commercial superglue formulation (Krazy glue, Elmer's Products Inc. Columbus Ohio). A 5000 ppm of a dichloro-fluorescein compound is mixed into the superglue and the properties of the cured films are compared to control films of the base polymer formulation. FIG. 9 is a stress-strain curves of a control sample of KrazyGlue+5% TOC 12 hrs (solid circles) and a sample of KrazyGlue+5% TOC with 5000 ppm dichlorofluorescen 12 hrs (open circles). The xanthenes-based compound exhibit good plasticizing performance in methyl cyanoacrylate. The methyl cyanoacrylates films are a stronger, or in other words can tolerate higher energy before breaking than the butyl cyanoacrylates.

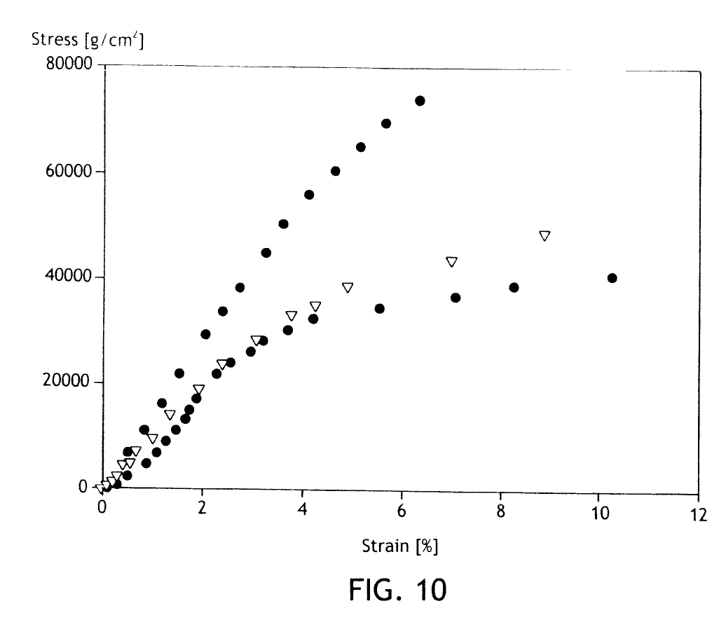

The superglue films exhibited higher stress-strain tolerances than the INTEGUSEAL™ films before rupturing because of the ethyl vs. butyl akyl structure. Methyl cyanoacrylates have relatively low tolerance to shear forces, while the plasticized INTEGUSEAL™ formulation is more able to tolerate shear forces. FIG. 10 shows the comparative stress-strain curves of the control sample of KrazyGlue +5% TOC 12 hrs. (solid circles); a sample of KrazyGlue+5% TOC with 5000 ppm dichlorofluorescein 12 hrs. (open circles), and sample of INTEGUSEAL™ with 5000 ppm dichlorofluorescein 12 hrs. (triangles).

### EXAMPLE

### Ethyl Cyanoacrylate

Ten 1-gram samples of superglue (Krazyglue, Elmer's Products, Inc., Columbus Ohio) containing ethyl cyanoacrylate are prepared. Test and control samples, respectively, with and without dichloroflurescein in the formulation were cured on microscope slides. Dichloroflurescein was present in the test samples at a concentration of 5000 ppm. The xanthenes dye-containing samples are observed to be relatively more flexible (i.e., plasticized) when compared to control samples. It appears that the alkyl group has no effect on the ability of the xanthenes to plasticize alkyl cyanoacrylates.

The present invention has been described both generally and in detail by way of examples and the figures. Persons skilled in the art, however, can appreciate that the invention is not limited necessarily to the embodiments specifically disclosed, but that substitutions, modifications, and variations may be made to the present invention and its uses without departing from the spirit and scope of the invention. Therefore, changes should be construed as included herein unless the modifications otherwise depart from the scope of the present invention as defined in the following claims.

We claim

1. A curable composition of matter comprising a semi-crystalline polymer with a minimal crystalline content of about 40% to about 55% by weight of the polymer, and a compound with a xanthene-based molecular structure in an amount up to about 5% and said composition exhibits a ratio of about 1.3:1.8:1.0 to about 1.6:1.5:1.0, respective of a mesophase:crystalline phase:amorphous phase when cured

2. The curable composition according to claim 1, wherein said composition exhibits a ratio of about 1.45:1.64:1.0, respective of said mesophase:crystalline phase:amorphous phase when cured.

3. The curable composition according to claim 1, wherein said polymer contains a crystalline content of about 35% to about 45% crystalline phase, 35% to about 60% mesophase, about 10% to about 27% amiior-phous state.

4. The curable composition according to claim 1, wherein said compound with a xanthene-based molecular structure is present in an amount of up to about 1.8 wt. %.

5. The cuLable composition according to claim 1, wherein said compound with a xanthenes-based molecular structure is present up to about 1.75 wt. %,

6. The curable composition according to claim 3, wherein said compound with a xanthene-based molecular structure is present at about 500 ppm (0.05 wt. %) to about 5000 ppm (0.5 wt. %).

7. The curable composition according to claim 1, wherein said semi-crystalline polymer is a vinylic fuictionalized monomer selected from: acrylate, cyanoacrylate, methacrylate, or styrene.

8. The curable composition according to claim 1, wherein said cyanoacrylate is an alkyl cyanoacrylate, wherein the alkyl group includes an ethyl, butyl, or propyl group.

9. The curable composition according to claim 1, wherein said semi-crystalline polymer is a copolymer derived from one or more cyanoacrylate monomers or a blend of cyanoacrylate monomers, wherein the cyanoacrylate monomers are selected from alkyl 2-cyanoacrylate, alkenyl 2-cyanoacrylate, alkoxyalkyl 2-cyanoacrylate, or carbalkoxyalkyl 2-cyanoacrylate, wherein the alkyl group may have 1 to 16 carbon atoms and may be methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate and 2-propoxyethyl 2-cyanoacrylate.

10. The curable composition according to claim 1, wherein said cyanoacrylate is a flexible skin sealant.

11. The curable composition according to claim 1, wherein said mesophase and said crystalline phase are each reduced by an amount of about 10-50% relative to the percentage of mesophase and crystalline phase of an identical composition absent said compound with xanthene-based molecular structure.

12. The curable composition according to claim 1, wherein when said composition is formed into a film with about 1 mm (±0.05 mm) thickness and cured, said composition exhibits, at a stress of 50,000 g/cm², a deformation of at least 40% greater than an identical composition absent said compound with xanthene-based molecular structure.

13. An article of manufacture comprising curable polymers, the curable polymer having a semi-crystalline polymer matrix incorporating a plasticizer comprising a xanthene molecule or a compound with a xanthene-based molecular structure.

14. The article according to claim 13, wherein said xanthene molecule or compound with a xanthene-based molecular structure is present in said polymer matrix in an amount of about 0.01 wt. % up to about 2.2 wt. %.

15. The article according to claim 13, wherein said xanthenes molecule or compound with a xanthenes-based molecular structure is present from about 0.03 wt. % up to about 1.8 wt. %,

16. The article according to claim 13, wherein said xanthenes molecule or compound with a xanthene-based molecular structure is present at about 500 ppm (0.05 wt. %) to about 5000 ppm (0.5 wt. %).

17. The article according to claim 13, wherein said semi-crystalline polymer is a vinylic functionalized monomer selected from: acrylate, cyanoacrylate, methacrylate, or styrene.

18. The article according to claim 23, wherein said semi-crystalline polymer is a copolymer derived from one or more cyanoacrylate monomers or a blend of cyanoacrylate monomers, wherein the cyanoacrylate monomers are selected from alkyl 2-cyanoacrylate, alkenyl 2-cyanoacrylate, alkoxyalkyl 2-cyanoacrylate, or carbalkoxyalkyl 2-cyanoacrylate, wherein the alkyl group may have 1 to 16 carbon atoms and may be methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate and 2-propoxyethyl 2-cyanoacrylate.

19. A method of plasticizing a crystalline-phase-containing polymer, the method comprises: providing in a mixture a polymer with about 30% to about 70% crystallinity and a plasticizing agent having a xantliene-based molecular structure present in an amount of up to about 2.2 wt. % of total composition; agitating and heating said mixture to a temperature of up to about 95° C.; and then allowing said mixture to cool to about ambient room temperature.

20. The method according to claim 19, wherein said mixture is heated to a temperature of between about 50° C. and 85° C.

21. The method according to claim 20, wherein said mixture is heated to a temperature of between about 60° C. and about 70° C.

22. The method according to claim 19, wherein said polymer initially has a mesophase of about 50% or less

23. A flexible barrier coating for mammalian skin, the coating comprising: a crystalline or semi-crystalline polymer and a plasticizing agent, said plasticizing agent having a xanthene or xanthene-based molecular structure, and said barrier coating exhibiting an elastic modulus approaching the elastic modulus of human skin.

24. The flexible barrier coating according to claim 23, wherein said barrier coating exhibits a modulus of about 1.8×10⁸ Pa to about 5.5×10⁸ Pa.

25. The flexible barrier coating according to claim 23, wherein said coating has a modulus of about 2×10⁸ Ida to about 4×10⁸ Pa.

26. The flexible barrier coating according to claim 23, wherein said coating is a skin sealant coating comprising: a curable semi-crystalline resin with a plasticizing agent.

27. The flexible barrier coating according to claim 23, wherein said semi-crystalline polymer is a vinylic functionalized monomer selected from: acrylate, cyanoacrylate, methacrylate, or styrene.

28. The flexible barrier coating according to claim 23, wherein said cyanoacrylate is an alkyl cyanoacrylate, wherein the alkyl group includes an ethyl, butyl, or propyl group.

29. The flexible barrier coating according to claim 23, wherein said semi-crystalline polymer is a copolymer derived from one or more cyanoacrylate monomers or a blend of cyanoacrylate monomers, wherein the cyanoacrylate monomers are selected from alkyl 2-cyanoacrylate, alkenyl 2-cyanoacrylate, alkoxyalkyl 2-cyanoacrylate, or carbalkoxyalkyl 2-cyanoacrylate, wherein the alkyl group may have 1 to 16 carbon atoms and may be methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate and 2-propoxyethyl 2-cyanoacrylate.
